(19) United States
(12) Patent Application Publication
(10) Pub. No.: US 2012/0142654 A1
(43) Pub. Date: Jun. 7, 2012

(54) PROGNOSTIC MARKER FOR MAMMA CARCINOMA

(75) Inventors: Thilo Doerk-Bousset, Hannover (DE); Michael Heuser, Hannover (DE); Frederik Martin Georges Damm, Paris (FR); Arnold Ganser, Hannover (DE); Tjoung-Won Park-Simon, Hannover (DE); Peter Hillemanns, Hannover (DE)

(73) Assignee: Medizinische Hochschule Hannover, Hannover (DE)

(21) Appl. No.: 13/312,536

(22) Filed: Dec. 6, 2011

(30) Foreign Application Priority Data
Dec. 6, 2010 (EP) 10193883.5
Dec. 7, 2010 (EP) 10194061.7

(51) Int. Cl. A61K 31/566; A61P 35/00; A61K 31/4196; C12Q 1/68; A61K 31/135

(52) U.S. Cl. 514/177; 435/6.11; 514/648; 514/383

(57) ABSTRACT

The invention provides an analytical method for the determination of the susceptibility of a human patient suffering from breast cancer to anti-cancer treatment by providing an analytical method for determining the genetic predisposition of a patient in respect of susceptibility, e.g. responsiveness, to anti-cancer treatment, especially treatment using at least one chemotherapeutical agent and/or at least one anti-hormonal agent on the basis of analysis of a genetic marker.

### PRIORITY CLAIM AND REFERENCE TO RELATED APPLICATION

This application claims priority under 35 U.S.C. §119 from European patent application No. 10193883.5, filed on Dec. 6, 2010, and of European patent application No. 10194061.7, filed on Dec. 7, 2010.

### FIELD

A field of the invention is anti-cancer treatement. The invention particularly concerns a prognostic marker in breast cancer, and to the analysis, and to the genetic marker, respectively, as an analyte for use in the selection of a therapy, as well as to an analytical method identifying the marker in breast cancer patients, especially for use in the prediction of the suitability of a treatment for a selected sub-group of patients.

### BACKGROUND

Hollink et al., Blood 5951-5960 (2009) found that there is no relation of the status of the marker Ref SNP ID rs16754 to the course of cancer in patients, because the alleles of the marker Ref SNP ID rs16754 have no influence on the amino acid sequence of the protein produced from the WT1 gene concerned.

Li and Man, Cancer Biomark. 109-116 (2009) describe that the expression of the WT1 gene is changed in breast cancer cells. Miyoshi et al., Clin. Cancer Res. 1167-1171 (2002) relate the expression level of the WT1 gene to a poor prognosis in breast cancer patients.

Arpino et al., Breast Cancer Res. R149-156 (2004) found that invasive lobular breast cancer was more likely to occur in older patients, larger in size, positive for estrogen receptor and for progesterone receptor, to have a lower S-phase fraction, to be negative for HER-2, p53, and EGF-receptor.

### SUMMARY OF THE INVENTION

The invention provides an analytical method for the determination of the susceptibility of a human patient suffering from breast cancer to anti-cancer treatment, e.g. by providing an analytical method for determining the genetic predisposition of a patient in respect of susceptibility, e.g. responsiveness, to anti-cancer treatment, especially treatment using at least one chemotherapeutical agent and/or at least one anti-hormonal agent.

The invention provides a method of analysis for identifying especially lobular breast cancer patients in respect of the prognosis of a treatment. The identification of these patients and the prognosis is based on the analysis of the allele of markers of the human WT1 gene, especially of the marker Ref SNP ID rs16754 and/or of a marker coupled to SNP ID rs16754. In addition to the analytical determination of patients for a specific therapy on the basis of the analysis of the genetic marker, the invention provides for the selection of patients on the basis of the analysis of the genetic marker for the therapy and for the therapeutic treatment of a group of patients bearing the allele of the marker as specified herein.

### BRIEF DESCRIPTION OF THE DRAWINGS

The invention is now described in greater detail with reference to the figures showing graphical representations of analytical results between breast cancer patients who are homozygous for the major allele (A/A) or have at least one minor allele (A/G and G/G) of the marker Ref SNP ID rs16754, namely in

FIG. 1 the OS in univariate analysis of all breast cancer patients (n=8094) analyzed,

FIG. 2 the OS for lobular breast cancer patients only,

FIG. 3 the breast cancer specific survival in lobular breast cancer patients,

FIG. 4 the OS for ER positive arid/or PR positive patients,

FIG. 5 the OS for ER negative and/or PR negative patients,

FIG. 6 the OS for lobular breast cancer patients younger than 50 years, and in

FIG. 7 the OS for lobular breast cancer patients older than 50 years according to the status of Ref SNP ID rs16754.

In the figures, year 0 of the time scale denotes the initial diagnosis and/or onset of cancer treatment.

### DETAILED DESCRIPTION OF PREFERRED EMBODIMENTS

The invention provides a diagnostic marker that is predictive of the suitability of a therapeutic treatment a human suffering from lobular breast cancer. Preferably, the diagnostic marker is independent from known diagnostic findings.

An embodiment of the invention provides an analytical method for use for the identification of breast cancer patients who can be treated successfully by standard therapy and for the identification of breast cancer patients who have a higher risk of relapse, e.g. 4 to 10 years after an initial cancer treatment, and who are therefore in need of additional treatment during this period. Accordingly, an embodiment of the invention provides a pharmaceutical composition for use as a medicament for treatment against the cancer in the patients that have a high risk of relapse, e.g. as a medicament for treatment within 4 to 10 years after an initial cancer treatment.

Embodiments of the invention relate to the analysis of a genetic marker for use as a prognostic marker in breast cancer, and to the analysis, and to the genetic marker, respectively, as an analyte for use in the selection of a therapy, as well as to an analytical method identifying the marker in breast cancer patients, especially for use in the prediction of the suitability of a treatment for a selected sub-group of patients. Further, the invention relates to the use of therapeutic agents for the long-term treatment, e.g. in a period of at least 3 to 5 years following initial cancer treatment or cancer diagnosis, especially in a period of at least 4 to 10 years following initial cancer treatment or cancer diagnosis, to improve the long-term treatment of breast cancer in a selected sub-group of patients, who are at least heterozygous for the genetic marker. The invention is based on the finding that in lobular breast cancer patients. one allele of the genetic marker is correlated with a poorer treatment prognosis, and hence the sub-group of patients requiring the administration of pharmaceutically active compounds as a medicament for treatment and/or prevention of lobular breast cancer, especially as a medicament for the prevention of recurring lobular breast cancer is characterized by the at least heterozygous presence of one allele of the marker. Interestingly, both alleles of the marker used in the invention give the same translation product, making the alleles a silent mutation.

In greater detail, the invention provides the use of the marker Ref SNP ID rs16754 of the human WT1 gene, which is located on chromosome 11p15 (Wilms' tumour 1 gene) as an indicator in the diagnosis of nucleic acid samples originating from breast cancer patients, especially of lobular breast cancer patients, more especially in hormone receptor positive patients, as it has been found that the presence of at least one minor allele of this marker correlates with an increased risk of relapse in these patients. Further, the invention provides for the use of additional genetic markers in these embodiments, as these additional markers have been found to be coupled to the marker Ref SNP ID rs16754 of the human WT1 gene. Accordingly, for the purposes of the invention, the reference to the marker Ref SNP ID rs16754 (SEQ ID NO: 5) includes the reference to the group comprising or consisting of coupled markers, which are given in Table 1. The sequences given for the markers including the markers of Table 1 denote the markers in their natural sequence environment as obtainable from NCBI at www.ncbi.nlm.nih.gov/snp/.

TABLE 1
Markers coupled to the marker Ref SNP ID rs16754

| | chromosome | | | | minor | major |
|---|---|---|---|---|---|---|
| marker | position | D′ | r² | LOD | allele | allele |
| rs5030277 | 32374245 | 1 | 0.88 | 17.86 | A | T |
| (SEQ ID NO: 6) | | | | | | |
| rs5030280 | 32374008 | 1 | 0.876 | 17.33 | C | T |
| (SEQ ID NO: 9) | | | | | | |
| rs5030281 | 32373451 | 1 | 0.881 | 18.17 | T | C |
| (SEQ ID NO: 7) | | | | | | |
| rs5030310 | 32367816 | 1 | 0.882 | 18.32 | T | A |
| (SEQ ID NO: 8) | | | | | | |
| rs11031755 | 32351574 | 1 | 0.882 | 18.32 | T | C |
| (SEQ ID NO: 10) | | | | | | |
| rs11031747 | 32343797 | 0.933 | 0.816 | 15.67 | C | T |
| (SEQ ID NO: 11) | | | | | | |
| rs3858439 | 32343490 | 0.936 | 0.824 | 16.62 | C | A |
| (SEQ ID NO: 12) | | | | | | |
| rs3858438 | 32343130 | 0.936 | 0.824 | 16.62 | G | C |
| (SEQ ID NO: 13) | | | | | | |
| rs11031746 | 32342995 | 0.936 | 0.824 | 16.62 | C | A |
| (SEQ ID NO: 14) | | | | | | |
| rs11031745 | 32342895 | 0.936 | 0.824 | 16.62 | T | C |
| (SEQ ID NO: 15) | | | | | | |
| rs11031744 | 32342815 | 0.936 | 0.824 | 16.62 | T | C |
| (SEQ ID NO: 16) | | | | | | |
| rs3858436 | 32342153 | 1 | 0.875 | 16.89 | G | A |
| (SEQ ID NO: 17) | | | | | | |
| rs2418901 | 32341389 | 1 | 0.823 | 16.44 | G | A |
| (SEQ ID NO: 18) | | | | | | |

In Table 1, the chromosome position of coupled markers relates to the nucleotide position on human chromosome 11 (genome build 36.3), on which the chromosome position of marker rs16754 at nt 32374521 is a reference, in relation to which the positions of markers coupled to rs16754 are located on the WT1 gene. D′ is the linkage disequilibrium (D), r is the correlation coefficient, and LOD is the LOD score estimating the recombination frequency. The alleles of the markers of table 1 that are coupled to the minor allele of marker rs16754, due to the coupling to rs16754 are also termed minor alleles and included in the term minor allele.

Accordingly, for the purposes of the invention, reference to the minor allele of the marker Ref SNP ID rs16754 includes the group comprising or consisting of minor alleles of the coupled markers of table 1. Accordingly, the group comprising, preferably consisting of rs16754 and the coupled markers of table 1, especially their minor alleles form one group of markers which are indicative of lobular breast cancer patients, especially node-positive patients, who are in need of a more intense, especially a more extended treatment period, e.g. during or following 4 to 10 years after initial treatment or diagnosis, which group of markers is located at positions 32374245 to 32341389 on human chromosome 11 (genome build 36.3), wherein the members of the group of markers are closely coupled to one another, especially to marker rs16754 at position 32374521.

From Table 1, the following markers are preferred for the embodiments of the invention in addition and/or in the alternative to Ref SNP ID rs16754: rs16754, rs5030277, rs5030280, rs5030281, rs5030310, and/or rs11031755, the minor alleles of which are very closely coupled the minor allele of Ref SNP ID rs16754.

It has been found that the at least heterozygous allele G of the marker Ref SNP ID rs16754 is an indicator of a poor diagnosis in breast cancer patients, especially in lobular breast cancer patients, and therefore is an indicator identifying patients in need of intense cancer therapy and/or in need of long-term therapy, e.g. in the period of e.g. 4 to 15 years, especially 5 to I 0 years after an initial cancer treatment, leading to a reduction in the proportion of relapse or progression rate of cancer. Accordingly, the analytical method of the invention allows to identify patients with the homozygous major allele of the marker Ref SNP ID rs16754 for less severe cancer treatment, because this group has a generally better prognosis, especially for long-term survival, whereas the analytical method allows to identify those patients who are in need of a more intense and/or more extended treatment, especially 4 to 10 years after initial cancer treatment, who are the patients having at least one minor allele of the marker Ref SNP ID rs16754.

In the process for analysis, the DNA from a breast cancer patient, preferably a node-positive lobular breast cancer patient, is analysed to determine the alleles of the marker Ref SNP ID rs16754 and/or of at least one of the markers coupled to Ref SNP ID rs16754, which coupled markers are given herein. As a result of the process, the absence or presence of at least one minor allele of at least one of the markers is determined.

It is a specific advantage of the analytical method of the invention to allow the identification of these groups of patients on the basis of a parameter, namely on the basis of the presence of the minor allele of the marker Ref SNP ID rs16754, which is independent from other analytical parameters, e.g. hormone receptor status (especially independent form the status of one of ER, PR and HER2), nodal status, tumour grade, and age in multivariate analysis.

Accordingly, the invention provides an analytical method determining the alleles of the marker Ref SNP ID rs16754 in a lobular breast cancer patient, e.g. followed by the step of selecting a method of treatment in dependence on the status of alleles of the marker Ref SNP ID rs16754, which status preferably is the at least heterozygous presence of the G-allele of the marker Ref SNP ID rs16754. Further, the invention provides pharmaceutical compositions for use as a medicament, i.e. for use as an active therapeutic agent in a pharmaceutical composition, especially a hormone receptor blocking agent, in the treatment of lobular breast cancer patients, which patients are characterized by the at least heterozygous allele G of the marker Ref SNP ID rs16754, e.g. as determined from a nucleic acid sample, e.g. total DNA obtained from a cell-containing sample of the patient.

Preferably, the presence of alleles of the marker Ref SNP ID rs16754 is determined by an analytical method comprising the step of amplification of a DNA section from a DNA sample obtained from the patient, preferably a total genomic DNA sample of the patient, e.g. by PCR, optionally with the step of analysing the nucleic acid sequence of the amplificate for identifying the nucleotide present in marker Ref SNP ID rs16754, e.g. the presence of the allele G, also termed minor allele or the presence of the allele A, which is also termed major allele, e.g. by DNA sequencing. Accordingly, the invention also relates to the use of a DNA amplificate generated in vitro by amplification of a DNA section of a patient's nucleic acid, e.g. a total DNA preparation or a total nucleic acid preparation isolated from a sample of the patient, for use as an indicator for identifying a sub-group of lobular breast cancer patients, who e.g. would benefit from or require intense cancer therapy and/or long-term therapy, e.g. in the period of e.g. 4 to 15 years, especially more than 5 or 6 years to 10 years after an initial cancer treatment. Preferably, the amplificate of the patient DNA containing the marker Ref SNP ID rs16754 for use as a diagnostic indicator, e.g. as a diagnostic indicator substance identifying a lobular breast cancer patient, especially as a patient requiring intense cancer therapy and/or in need of long-term therapy, e.g. in the period of e.g. 4 to 15 years, especially 5 to 10 years after an initial cancer treatment, is an amplificate having e.g. a length of 10 to 1500 bp, and is generated by PCR using at least one pair of primers which is specific for the DNA section. Specific primer pairs for amplification of a DNA section from total patient DNA, e.g. total genomic DNA, can be designed by standard methods, and processes for identifying the allele of marker Ref SNP ID rs16754 as G and/or A are also generally known, e.g. DNA sequencing or specific hybridisation of the amplificate containing the marker, which amplificate was obtained from the genomic DNA as a template for PCR. The marker including the reference number Ref SNP ID rs16754 is e.g. available at NCBI databank (build 129), and is also termed C1107 A>G at GenBank entry NM/024426.3 (NM-024426.3: c.1107 A>G). The nucleotide sequence of the wt1 gene according to NM024426 is enclosed as SEQ ID NO: 1, wherein nucleotides 197 to 1750 contain coding sequence, with exon 7 at nucleotides 1295 to 1445, and marker Ref SNP ID rs16754 at position 1303, which corresponds to nucleotide No 1107 of the coding sequence section (nt 197 to 1750). The amino acid sequence encoded by exon 7 is given as SEQ ID NO: 2, irrespective of the natural reading frame.

Preferably, the analytical method of the invention further includes at least one additional analytical step for analysis of the lobular breast cancer, which analytical step is selected from the group comprising or consisting of an analysis

in respect of its estrogen receptor status, e.g. negative or positive, as can be determined by standard analytical processes, e.g. by immuno histochemistry,
in respect of its progesteron receptor status, e.g. negative or positive as can be determined by standard analytical processes, e.g. by immuno histochemistry,
in respect of its lymph node (node) status, e.g. node negative or node positive as can be determined by standard analytical processes, e.g. by histopathology,
in respect of its HER-2 status, e.g. negative or positive as can be determined by standard analytical processes, e.g. by immuno histochemistry,
in respect of its tumour grade status, e.g. grade 2 vs. grade 1 and grade 3 vs. grade 1, e.g. can be determined by pathologic evaluation,
and in respect of patient age, e.g. younger than 50 years vs. 50 years and older.

It has been found that especially patients with lobular breast cancer who at least heterozygously have the minor allele of the marker Ref SNP ID rs16754 who additionally have a positive hormone receptor status, e.g. selected from estrogen receptor status, progesteron receptor status and/or HER-2 status have a significantly worse prognosis, and are therefore in need for additional therapy, e.g. in the period of 4 to 10 years following initial cancer treatment. Accordingly, these patients are the preferred patients of the invention, e.g. lobular breast cancer patients carrying at least one minor allele of the marker Ref SNP ID rs16754 and having a tumour that is positive for hormone receptor status, e.g. selected from estrogen receptor status, progesteron receptor status and/or HER-2 status, especially estrogen receptor status and progesteron receptor status.

### Medical Studies

The present invention is based on a large-scale medical study involving 8337 female breast cancer patients, in whom the alleles of the marker Ref SNP ID rs16754 were analysed in PBMC (peripheral mononuclear blood cells) and relevance of the alleles was assessed in a univariate and multivariate analyses considering age, tumour grade, lymph node involvement (nodal status), estrogen receptor status, progesterone receptor status, and HER-2 status. Generally, the data that were analysed originate from patients undergoing similar treatments, e.g. treatment was similar, irrespective of lobular or ductal breast cancer, and irrespective of the alleles of the marker Ref SNP ID rs16754.

It was found that the minor allele of the marker Ref SNP ID rs16754 is heterozygous in 25.2% of the patients and homozygous in 2.8% of the patients. Patient characteristics and pathological findings were similarly distributed between patients who are homozygous for the major allele (A/A) of the marker Ref SNP ID rs16754, and those patients who are at least heterozygous for the minor allele of the marker Ref SNP ID rs16754, i.e. heterozygous (A/G) or homozygous (G/G) for the minor allele (G).

Only in patients (n=1101) having lobular breast cancer, it was determined that the alleles of the marker Ref SNP ID rs16754 were independently predictive for overall survival (OS) in multivariate analysis (HR 1.44, 95% C1 1.06-1.94, P=0.018), and for breast cancer specific survival with HR 1.49, 95% C1 1.07-2.07, P=0.018, wherein HR is the hazard ratio, C1 is the confidence interval, and P is the P-value indicating significance, whereas in other histological subtypes, e.g. in ductal breast cancer, a predictive value of the marker Ref SNP ID rs16754 was not found. The linkage of a minor allele of the marker Ref SNP ID rs16754 with a poor prognosis, and the need for more intense and/or longer administration of anti-cancer medicaments became more pronounced at 4 or 5 years after initial diagnosis and/or after initial treatment, and was most pronounced in hormone receptor positive and in younger lobular breast cancer patients.

Preferably, compounds for use as a medicament in the treatment of lobular breast cancer patients having at least one minor allele of the marker Ref SNP ID rs16754 are selected from compounds used as medicaments in the treatment of hormone receptor positive breast cancer patients, especially tamoxifen, ovarian suppression agents, aromatase inhibitors, and irreversible estrogen receptor inhibitors or combinations of these, especially Tamoxifen, Anastrozol, Letrozol, and Exemestan.

### EXAMPLE 1

### Analysis of the Marker Ref SNP ID rs16754

Analyses of the alleles of the marker Ref SNP ID rs16754 were performed using the commercially available (Applied Biosystems, Assay ID C_{—}12034416_{—}10) PCR-based assay specific for marker Ref SNP ID rs16754. Generally, total DNA was isolated from PBMC of breast cancer patients and subjected to PCR in microliter plates at an annealing temperature of 60° C. using the TaqMan protocol supplied by the manufacturer. For amplification, a forward primer of SEQ ID NO 3 and a backward primer of SEQ ID NO 4 were used. Detection and assessment was made with the Sequence Detection Software supplied by the manufacturer.

For statistical analyses, median follow-up time for survival was calculated according to Korn, Stat Med 5: 255-260 (1986). Overall survival (OS) endpoints were death (failure) and alive at last follow-up (generally censored at 10 years), measured from the date of entry into the study. Pair-wise comparisons of variables for exploratory purposes were done using the Kolmogorov-Smirnov test for continuous variables and the Chi-squared test for categorical variables. Estimation of distribution of OS and comparison of differences between survival curves was by the Kaplan-Meier method and the log-rank test, respectively. A Cox proportional hazards model for OS was developed for multivariate analysis and stratified by clinical study. The proportional hazards assumption was checked by scaled Schoenfeld residuals. Variables with P of 0.1 or below in univariate analysis were included into the model. Status of the marker Ref SNP ID rs16754 (homozygous for major allele vs. at least one minor allele), nodal status (node positive vs. node negative), estrogen receptor (ER) status (ER positive vs. ER negative), progesterone receptor (PR) status (PR positive vs. PR negative), HER-2 status (HER-2 positive vs. HER-2 negative), tumour grade (grade 2 vs. grade 1, and grade 3 vs. grade 1), and age (below 50 years vs. at least 50 years) were the variables considered.

Incomplete (more than 60%) data sets of patients were used for the multivariate Cox model following imputation of missing data using the aregImpute function of the R software package Hmisc, version 3.7, allowing the algorithm to use data from all other variables, except for the age. This procedure models the distribution of each variable present to the other variables with a predictive mean matching, and using bootstrapping to generate multiple imputations by approximating samples from the posterior predictive distributions of missing values to account for all aspects of uncertainty. Following an initial 10 data sets, a further 50 data sets were imputed. Using a logistic regression model, associations between variables and response to induction therapy were analysed. For relevance of quantitative results, odds ratios (OR) and hazard ratios (HR) were calculated at 95% C1, and the two-sided level of significance was set at P less than 0.05. For statistical analyses, the SPSS programme version 17.0 and R programme version 2.10 were used.

It was found that 71.9% of patients were homozygous for the major allele of the marker Ref SNP ID rs16754 (A/A), 25.2% were heterozygous (A/G), and 2.8% were homozygous for the minor allele of the marker Ref SNP ID rs16754 (G/G). Generally, this allele distribution corresponds to that of the general European population. When comparing pre-treatment clinical and molecular characteristics of the patients, including age, bilateral disease, tumour stage and grade, nodal status, tumour histology, ER status, PR status, ER/PR status, HER-2 status or ER/PR/HER-2 status, no significant differences were found between patients who are homozygous for the major allele to patients having at least one minor allele of the marker Ref SNP ID rs16754.

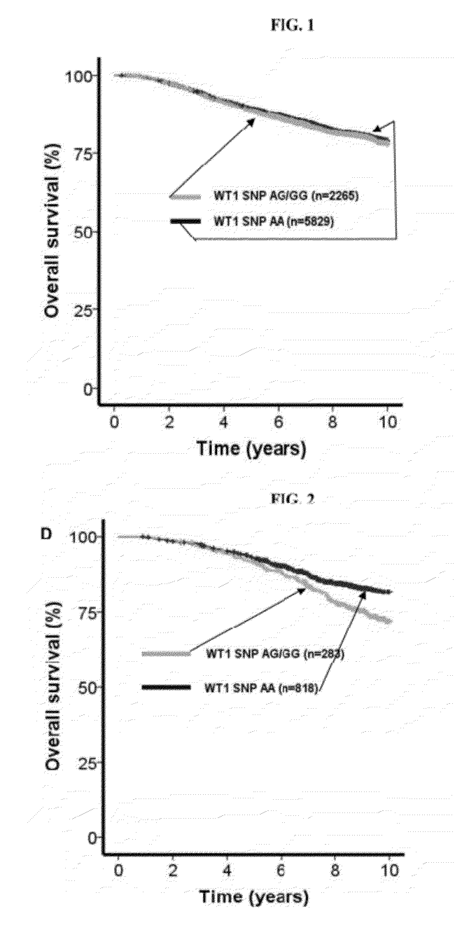

When analysing overall survival (average 9.67 years, range 0.3 to 10 years) of all breast cancer patients (n=8094; P=0.54), no significant difference was found between homozygous major alleles and at least heterozygous minor alleles of the marker Ref SNP ID rs16754, as shown in FIG. 1. In ductal carcinoma patients, OS was not significantly different between patients with at least one minor allele and patients homozygous for the major allele (lymph node negative P=0.54, lymph node positive P=0.46, ductal carcinoma P=0.65) Further, OS was not significantly different in node positive (n=2825) patients between patients homozygous for the major allele and patients having at least one minor allele of the marker Ref SNP ID rs16754 (P=0.41).

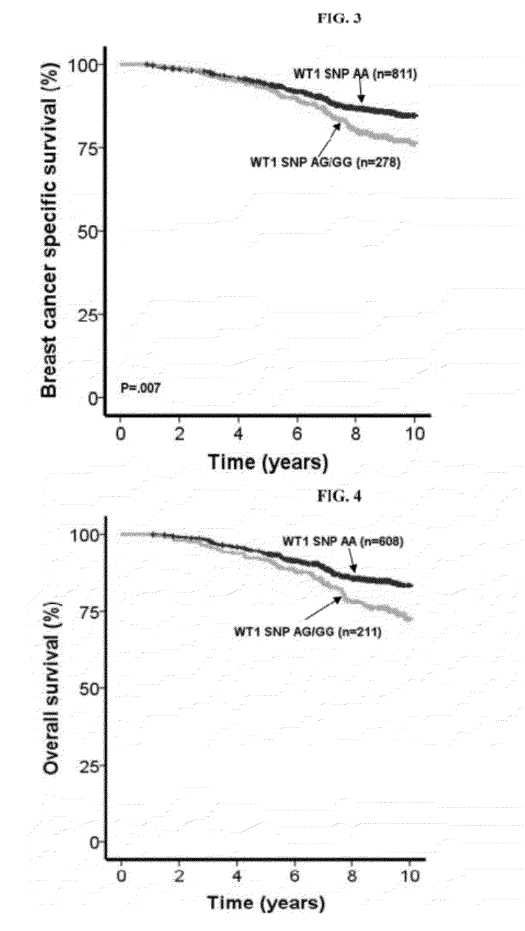

However, it was found that OS of lobular breast cancer patients having at least one minor allele of the marker Ref SNP ID rs16754 was significantly shorter (HR=1.54, 95% C1 1.15-2.06, P=0.004) compared to lobular breast cancer patients having the major allele of the marker Ref SNP ID rs16754 homozygously, as shown in FIG. 2. Lobular breast cancer patients having at least one minor allele of the marker Ref SNP ID rs16754 showed a 5-year OS of 88% and a 10-year OS of 71% compared to a 5-year OS of 90% and a 10-year OS of 81% for lobular breast cancer patients having the major allele of the marker Ref SNP ID rs16754 homozygously. It was found that the baseline characteristics were similarly distributed between patients homozygous for the major allele and patients having the minor allele of the marker Ref SNP ID rs16754, respectively, except for a higher occurrence of node positive breast cancer in patients with at least one minor allele (41% vs. 35%, P=0.058). Importantly, BCSS was significantly shorter in lobular breast cancer patients with at least one minor allele of SNP rs16754 compared to major allele homozygous patients (HR 1.55, 95% C1 1.13-2.14, P=0.007) as shown in FIG. 3.

In multivariate analysis of variables having P=0.1 or below in univariate analysis, it was found that presence of at least one minor allele of the marker Ref SNP ID rs16754 in lobular breast cancer patients is associated and predictive of an increased hazard of death (HR 1.46, 95% C1 1.08-1.96, P=0.013). Details are shown in the following Table 2, wherein HR above 1 indicates an increased risk for the respective variable, and HR below 1 indicates a decreased risk.

TABLE 2
Multivariate analysis for OS in lobular
breast cancer patients (n = 1089)

| Variable | HR | 95% CI | P |
|---|---|---|---|
| at least one minor allele of the | 1.49 | 1.07-2.07 | 0.018 |
| marker Ref SNP ID rs16754 vs. | | | |
| homozygousity for major allele of | | | |
| the marker Ref SNP ID rs16754 | | | |
| node positive vs. node negative | 3.24 | 2.27-4.62 | <0.001 |
| ER positive vs. ER negative | 0.82 | 0.48-1.41 | 0.47 |
| PR positive vs. PR negative | 0.58 | 0.39-0.88 | 0.01 |
| HER-2 positive vs. HER-2 negative | 1.24 | 0.54-2.85 | 0.62 |
| grade 2 vs. grade 1 | 1.42 | 0.9-2.23 | 0.13, |
| grade 3 vs. grade 1 | 2.52 | 1.31-4.83 | 0.006 |
| age ≧50 years vs. age <50 years | 0.73 | 0.51-1.05 | 0.09 |

Herein, a hazard ratio (HR) above 1 indicates an increased risk for the first category listed.

### EXAMPLE 2

### Medicament for Treatment of Lobular Breast Cancer in Patients having a Minor Allele of the Marker Ref SNP ID rs16754

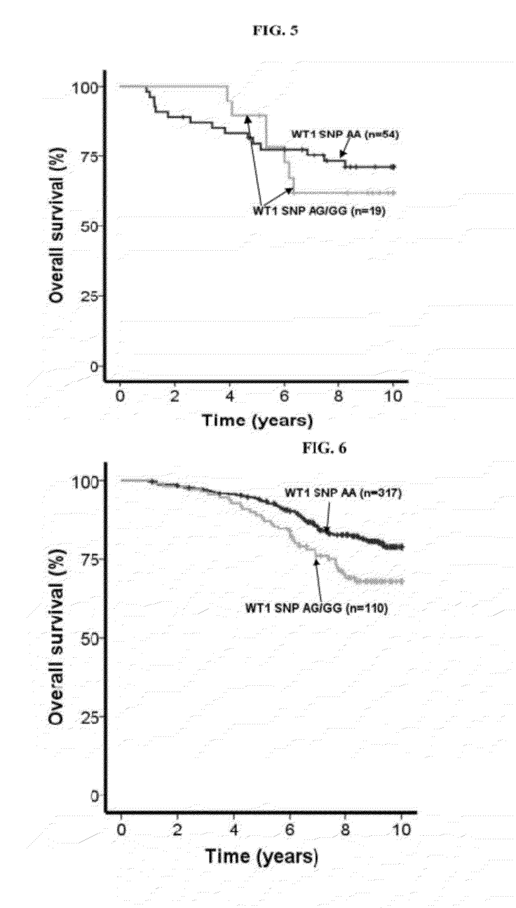

In the data analysis it became clear that the presence of at least one minor allele of the marker Ref SNP ID rs16754 in lobular breast cancer patients especially in a period of approx. 4-5 years after initial cancer diagnosis and treatment is correlated with a negative prognosis, i.e. a lower OS. When analysing the correlation of the alleles of the marker Ref SNP ID rs16754 to hormone receptor positive and negative patients, it was found that the presence of the minor allele was correlated with a reduced OS in hormone receptor positive patients (P=0.004, FIG. 4), but not in hormone receptor negative patients (P=0.75, FIG. 5).

In contrast to the hormone negative patients, the hormone receptor positive patients were generally treated with a hormone ablation therapy, which typically involves the administration of Tamoxifen. From the correlation of the presence of the minor allele of the marker Ref SNP ID rs16754 in lobular breast cancer patients with a reduced OS in those patients who due to the negative hormone receptor status generally did not receive treatment in the period 5 years after initial diagnosis, and the observation that presence of the minor allele is not correlated with a reduced OS in patients that receive treatment hormone ablation treatment, it can be concluded that lobular breast cancer patients having at least one minor allele of the marker Ref SNP ID rs16754 benefit from treatment with anti-hormone receptor pharmaceutical compounds irrespective of the presence of infiltrated lymph nodes.

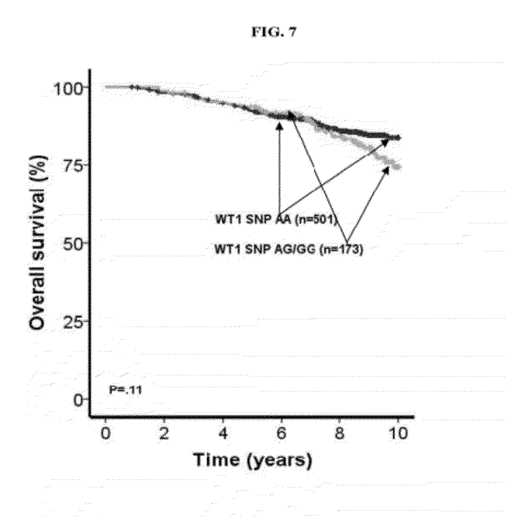

The above finding of a positive therapeutic effect of hormone ablation therapeutics in lobular breast cancer patients having at least one minor allele of the marker Ref SNP ID rs16754 was confirmed in menopausal patients. Statistical analysis showed the correlation of the presence of at least one minor allele of the marker Ref SNP ID rs16754 with reduced OS in younger patients (age of less than 50 years, mean age 43.7 years), who generally do not receive hormone ablation therapy, compared to patients who are homozygous for the major allele of the marker Ref SNP ID rs16754 (P=0.01, FIG. 6). The difference in OS was less evident in older patients (age above 50 years, mean age 59.1 years), who generally receive hormone ablation therapy, between lobular breast cancer patients having at least one minor allele of the marker Ref SNP ID rs16754 and patients who are homozygous for the major allele of the marker Ref SNP ID rs16754 (P=0.13, FIG. 7).

Hence, the invention also provides for compounds, e.g. selected from the group of pharmaceutical compounds used in hormone ablation therapy, e.g. a hormone receptor blocking agent, especially Tamoxifen, Anastrozol, Letrozol, Exemestan, for use as a medicament in the treatment of lobular breast cancer patients having at least one minor allele of the marker Ref SNP ID rs16754, especially for administration in a period of time after 4 to 5 years, e.g. up to 10 years, following initial breast cancer diagnosis or following initial breast cancer treatment.

Further, the analysis of OS in node positive lobular breast cancer patients revealed no significant difference between patients being homozygous for the major allele of the marker Ref SNP ID rs16754 and patients having at least one minor allele of the marker Ref SNP ID rs16754. When considering that generally the presence of the minor allele of the marker Ref SNP ID rs16754 is correlated with a reduced OS, the finding that node positive breast cancer patients generally receive cancer treatment by irradiation and/or chemotherapy to a higher extent than node negative patients demonstrates that especially node positive lobular breast cancer patients having at least one minor allele of the marker Ref SNP ID rs16754 benefit from the additional or harsher cancer treatment, e.g. irradiation and/or chemotherapy. Hence, the invention also provides for compounds, e.g. selected from the group of pharmaceutical compounds used in cancer therapy, e.g. Tamoxifen, Anastrozol, Letrozol or Exemestan for use as a medicament in the treatment of lobular breast cancer patients having at least one minor allele of the marker Ref SNP ID rs16754 or having the coupled allele of a marker coupled to the marker Ref SNP ID rs16754, especially for administration in a period of time after 4 to 5 years following initial breast cancer diagnosis or treatment, especially those patients who are node positive and/or patients who are node negative.

While specific embodiments of the present invention have been shown and described, it should be understood that other modifications, substitutions and alternatives are apparent to one of ordinary skill in the art. Such modifications, substitutions and alternatives can be made without departing from the spirit and scope of the invention, which should be determined from the appended claims.

Various features of the invention are set forth in the appended claims.

1. Process for the analysis of the susceptibility of a human patient to anti-cancer treatment, comprising the steps of providing a nucleic acid sample obtained from a human suffering from lobular breast cancer and analysing the alleles of the marker Ref SNP ID rs16754 and/or of the alleles of at least one marker of the group of markers comprising rs5030277, rs5030280, rs5030281, rs5030310, rs11031755, rs11031747, rs3858439, rs3858438, rs11031746, rs11031745, rs11031744, rs3858436, rs2418901, in the nucleic acid sample obtained from a human suffering from lobular breast cancer.

2. Process according to claim 1, wherein the breast cancer has at least one positive hormone receptor status.

3. Process according to claim 2, wherein the positive hormone receptor status is at least one of estrogen receptor status, progesteron receptor status and/or HER-2 status.

4. Process according to claim 1, wherein the process is for use in the selection of a therapy against lobular breast cancer.

5. Process according to claim 1, wherein the nucleic acid sample is provided, and the alleles of the marker Ref SNP ID rs16754 and/or the alleles of at least one of the markers of the group comprising rs5030277, rs5030280, rs5030281, rs5030310, rs11031755, rs11031747, rs3858439, rs3858438, rs11031746, rs11031745, rs11031744, rs3858436, rs2418901 are determined, and the hormone receptor status of the patient is determined.

6. Process according to claim 1, wherein the hormone receptor status of the cancer is determined.

7. Use of a kit of parts containing oligonucleotides which are complementary to at least a section of the human WT1 gene corresponding to SEQ ID NO 1 as reagents for the analysis of the allele of the marker Ref SNP ID rs16754 and/or of the allele of at least one of the markers of the group comprising rs5030277, rs5030280, rs5030281, rs5030310, rs11031755, rs11031747, rs3858439, rs3858438, rs11031746, rs11031745, rs11031744, rs3858436, rs2418901 in a process according to claim 1 in a nucleic acid sample obtained from a human patient suffering from lobular breast cancer.

8. Method of treatment of a patient having lobular breast cancer by administration of a pharmaceutical composition containing Tamoxifen, Anastrozol, Letrozol or Exemestan as an active compound wherein the patient is analysed to bear the allele G in marker Ref SNP ID rs16754 at least heterozygously, or the minor allele of at least one of the markers of the group comprising rs5030277, rs5030280, rs5030281, rs5030310, rs11031755, rs11031747, rs3858439, rs3858438, rs11031746, rs11031745, rs11031744, rs3858436, rs2418901 at least heterozygously.

9. Method of treatment according to claim 8, wherein the treatment is for at least 5 to 10 years, preferably 6 years to 10 years following the detection of breast cancer.

10. Method of treatment according to claim 8, wherein the patients have received treatment against breast cancer 5 to 10 years previously.

11. Method of treatment according to one of claims 8, wherein the pharmaceutical composition containing a hormone receptor blocking agent as the pharmaceutically active compound.

12. Use of a process for analysis of the alleles of the marker Ref SNP ID rs16754 or of the alleles of at least one of the markers of the group comprising rs5030277, rs5030280, rs5030281, rs5030310, rs11031755, rs11031747, rs3858439, rs3858438, rs11031746, rs11031745, rs1103 1744, rs3858436, rs2418901 in a nucleic acid sample, wherein the nucleic acid sample was obtained from a human patient suffering from lobular breast cancer, in the selection of a therapy in a period of 3 to 10 years following the detection of breast cancer.

13. Use of an amplified section of the human WT1 gene, which section is obtainable by enzymatic DNA amplification from a DNA sample originating from a lobular breast cancer patient, which section comprises the marker Ref SNP ID rs16754 and/or at least one of the markers from the group comprising rs5030277, rs5030280, rs5030281, rs5030310, rs11031755, rs11031747, rs3858439, rs3858438, rs 11031746, rs11031745, rs11031744, rs3858436, rs2418901, as an indicator substance for identifying patients.

14. Use according to claim 13, wherein the patients are identified by the presence of at least one G-allele of the marker Ref SNP ID rs16754 and/or at least one minor allele of at least one of the markers of the group consisting of rs5030277, rs5030280, rs5030281, rs5030310, rs11031755, rs11031747, rs3858439, rs3858438, rs11031746, rs11031745, rs11031744, rs3858436, and rs2418901.

15. Use according to one of claims 13, wherein the amplified DNA section comprises nucleotides 32374245 to 32341389 of human chromosome 11.


SEQUENCE LISTING (18 sequences)

SEQ ID NO: 1 - DNA, 3029 bp, Homo sapiens
  CDS (197)..(1750): /gene=“exon 7” /note=“SNP ID rs16754”
ccaggcagctggggtaaggagttcaaggcagcgcccacacccgggggctctccgcaacccgaccgcctgtccgctcccccacttcccgccctccctcccacctactcattcacccacccacccacccagagccgggacggcagcccaggcgcccgggccccgccgtctcctcgccgcgatcctggacttcctcttgctgcaggacccggcttccacgtgtgtcccggagccggcgtctcagcacacgctccgctccgggcctgggtgcctacagcagccagagcagcagggagtccgggacccgggcggcatctgggccaagttaggcgccgccgaggccagcgctgaacgtctccagggccggaggagccgcggggcgtccgggtctgagccgcagcaaatgggctccgacgtgcgggacctgaacgcgctgctgcccgccgtcccctccctgggtggcggcggcggctgtgccctgcctgtgagcggcgcggcgcagtgggcgccggtgctggactttgcgcccccgggcgcttcggcttacgggtcgttgggcggccccgcgccgccaccggctccgccgccacccccgccgccgccgcctcactccttcatcaaacaggagccgagctggggcggcgcggagccgcacgaggagcagtgcctgagcgccttcactgtccacttttccggccagttcactggcacagccggagcctgtcgctacgggcccttcggtcctcctccgcccagccaggcgtcatccggccaggccaggatgtttcctaacgcgccctacctgcccagctgcctcgagagccagcccgctattcgcaatcagggttacagcacggtcaccttcgacgggacgcccagctacggtcacacgccctcgcaccatgcggcgcagttccccaaccactcattcaagcatgaggatcccatgggccagcagggctcgctgggtgagcagcagtactcggtgccgcccccggtctatggctgccacacccccaccgacagctgcaccggcagccaggctttgctgctgaggacgccctacagcagtgacaatttataccaaatgacatcccagcttgaatgcatgacctggaatcagatgaacttaggagccaccttaaagggagttgctgctgggagctccagctcagtgaaatggacagaagggcagagcaaccacagcacagggtacgagagcgataaccacacaacgcccatcctctgcggagcccaatacagaatacacacgcacggtgtcttcagaggcattcaggatgtgcgacgtgtgcctggagtagccccgactcttgtacggtcggcatctgagaccagtgagaaacgccccttcatgtgtgcttacccaggctgcaataagagatattttaagctgtcccacttacagatgcacagcaggaagcacactggtgagaaaccataccagtgtgacttcaaggactgtgaacgaaggttttctcgttcagaccagctcaaaagacaccaaaggagacatacaggtgtgaaaccattccagtgtaaaacttgtcagcgaaagttctcccggtccgaccacctgaagacccacaccaggactcatacaggtaaaacaagtgaaaagcccttcagctgtcggtggccaagttgtcagaaaaagtttgcccggtcagatgaattagtccgccatcacaacatgcatcagagaaacatgaccaaactccagctggcgctttgaggggtctccctcggggaccgttcagtgtcccaggcagcacagtgtgtgaactgctttcaagtctgactctccactcctcctcactaaaaaggaaacttcagttgatcttcttcatccaacttccaagacaagataccggtgcttctggaaactaccaggtgtgcctggaagagttggtctctgccctgcctacttttagttgactcacaggccctggagaagcagctaacaatgtctggttagttaaaagcccattgccatttggtgtggattttctactgtaagaagagccatagctgatcatgtccccctgacccttcccttctttttttatgctcgttttcgctggggatggaattattgtaccattttctatcatggaatatttataggccagggcatgtgtatgtgtctgctaatgtaaactttgtcatggtttccatttactaacagcaacagcaagaaataaatcagagagcaaggcatcgggggtgaatcttgtctaacattcccgaggtcagccaggctgctaacctggaaagcaggatgtagttctgccaggcaacttttaaagctcatgcatttcaagcagctgaagaaaaaatcagaactaaccagtacctctgtatagaaatctaaaagaattttaccattcagttaattcaatgtgaacactggcacactgctcttaagaaactatgaagatctgagatttttttgtgtatgtttttgactcttttgagtggtaatcatatgtgtctttatagatgtacatacctccttgcacaaatggaggggaattcattttcatcactgggagtgtccttagtgtataaaaaccatgctggtatatggcttcaagttgtaaaaatgaaagtgactttaaaagaaaataggggatggtccaggatctccactgataagactgtttttaagtaacttaaggacctttgggtctacaagtatatgtgaaaaaaatgagacttactgggtgaggaaatccattgtttaaagatggtcgtgtgtgtgtgtgtgtgtgtgtgtgtgtgtgtgttgtgttgtgttttgttttttaagggagggaatttattatttaccgttgcttgaaattactgtgtaaatatatgtctgataatgatttgctctttgacaactaaaattaggactgtataagtactagatgcatcactgggtgttgatcttacaagatattgatgataacacttaaaattgtaacctgcatttttcactttgctctcaattaaagtctattcaaaag

SEQ ID NO: 2 - protein, 517 aa, Homo sapiens
LQDPASTCVPEPASQHTLRSGPGCLQQPEQQGVRDPGGIWAKLGAAEASAERLQGRRSRGASGSEPQQMGSDVRDLNALLPAVPSLGGGGGCALPVSGAAQWAPVLDFAPPGASAYGSLGGPAPPPAPPPPPPPPPHSFIKQEPSWGGAEPHEEQCLSAFTVHFSGQFTGTAGACRYGPFGPPPPSQASSGQARMFPNAPYLPSCLESQPAIRNQGYSTVTFDGTPSYGHTPSHHAAQFPNHSFKHEDPMGQQGSLGEQQYSVPPPVYGCHTPTDSCTGSQALLLRTPYSSDNLYQMTSQLECMTWNQMNLGATLKGVAAGSSSSVKWTEGQSNHSTGYESDNHTTPILCGAQYRIHTHGVFRGIQDVRRVPGVAPTLVRSASETSEKRPFMCAYPGCNKRYFKLSHLQMHSRKHTGEKPYQCDFKDCERRFSRSDQLKRHQRRHTGVKPFQCKTCQRKFSRSDHLKTHTRTHTGKTSEKPFSCRWPSCQKKFARSDELVRHHNMHQRNMTKLQLAL

SEQ ID NO: 3 - DNA, 21 bp, Artificial Sequence
  /note=“SEQ ID NO 3, forward primer”
ctccagtgctcactctccctc

SEQ ID NO: 4 - DNA, 21 bp, Artificial Sequence
  /note=“SEQ ID NO 4, backward primer”
ccttagcagtgtgagagcctg

SEQ ID NO: 5 - DNA, 511 bp, Homo sapiens
  misc_difference (1)..(511): /note=“rs16754”
ttcatctcaacagccacttctggttgctcagtagtgatgatcttagagcactgcattgtagaatgtcctgatctagcatccattactgatgtctgccatggggatctggagtgtgaatgggagtggttttaggtctgcacctgccaccccttctttggatatactccagtgctcactctccctcaagacctacgtgaatgttcacatgtggcttaaagcctcccttcctcttactctctgcctgcaggatgtgcgrcgtgtgcctggagtagccccgactcttgtacggtcggcatctgagaccagtgagaaacgccccttcatgtgtgcttacccaggctgcaataagagatattttaagctgtcccacttacagatgcacagcaggaagcacactggtaagtgtgcccgctgtccagtcttgggcaaacatggttcaagagctccttttccaggctctcacactgctaaggacccaggttatttccaggatggaaagtaagcactgctt

SEQ ID NO: 6 - DNA, 511 bp, Homo sapiens
  misc_difference (1)..(511): /note=“rs5030277”
gactcttgtacggtcggcatctgagaccagtgagaaacgccccttcatgtgtgcttacccaggctgcaataagagatattttaagctgtcccacttacagatgcacagcaggaagcacactggtaagtgtgcccgctgtccagtcttgggcaaacatggttcaagagctccttttccaggctctcacactgctaaggacccaggttatttccaggatggaaagtaagcactgctttgaaaggtctgatccaggtgwtgtaaagaggctgtggggccattttgcattctgattatctatcctttattacagaggtcagtaaactgtcttccagaaaggcagagcagagtggtagagagaaagtgccacagaattaaagagacctgtgtaggaatcgtgttggtgttagaatactattagcatttctaaggagagaggagctggcctgggaatatagtcatcagatggttttaggaggtcgatgggcaagatcattgtgaaattttagaagag

SEQ ID NO: 7 - DNA, 511 bp, Homo sapiens
  misc_difference (1)..(511): /note=“rs5030281”
caggagaattgcttgaacctaggaggcagaggttgcagtgagctgagatcttaccactgtactccagcctaggcgacagagcaagactctgccaaaaaaaaaaaaaaggagagagagagatgatgattttttatctttgaagatacgccaatgaaactggattttcccccaggtcagagaggctgcttctgggaaacagagaactggcattgtttggtgttggccaccttgaatgtggacagccatgtcacagaaygtttactctgggccttgtgggcactaggtgggagtgtgggagtgtggtaaaacccaggttttcctacagaggagctactgctgccttttccacaatcgaagaacagcattattataggatcactgttgcaactgtgttattactgctgattttgctcttgggaagctggagaaaaaagtgagctgcattcagatttccttggaccattgaaatttgctaattcactgtgtccagaggatgcaattctggtgtcaa

SEQ ID NO: 8 - DNA, 511 bp, Homo sapiens
  misc_difference (1)..(511): /note=“rs5030310”
ccagcacaaggcttggcatatgttaagcatcaaataaatggtagttattctccctaatgagaatgaggcttggaaagaatgtctgagggctctttgggtggagaagcctcaatcttttggcaagcttattaaaaatctaagaaccacgccccaactagaatcttccatataacaggattatgctctcagtaacactttaagagaaaaaaaaaaacaacacataacatgtaaactggtatcagtgatttattttaawgtatccagcccatccagggtatctacacagatagaagtgttaaaagcttcagtcacagctgagatccctggacagtgatcactcagtaatacccagggtagatgacccagaggcttctgcagcccagttcatctcagttcccaacttactcaacaagggggagaaatttacacagttcacaaaacattctatcagcagagactgaaacaaacgtttttccccttaattatgaattgctcagacatttcccccaac

SEQ ID NO: 9 - DNA, 685 bp, Homo sapiens
  misc_difference (1)..(685): /note=“ | rs5030280”
atctatcctttattacagaggtcagtaaactgtcttccagaaaggcagagcagagtggtagagagaaagtgccacagaattaaagagacctgtgtaggaatcgtgttggtgttagaatactattagcatttctaaggagagaggagctggcctgggaatatagtcatcagatggttttaggaggtcgatgggcaagatcaytgtgaaattttagaagagatggtccaaagaaaaacttacctgcttgggtcagtaataatcaactagacaggaaggggaaaattgattgttagtttgattttttagtgatcacttaaaaaaaaaaggtcagtcatgggggctcacgcctgtaatcccagcactttgggaggccgaagtgggtggatcacccaaggtcaggagttggagaccagcctggccaacaaggtgaaaccccgtctctactaaaaatacaaaaatgagccgggtgtggtggcacgcacctgtacttgggaggctggggcaggagaattgcttgaacctaggaggcagaggttgcagtgagctgagatcttaccactgtactccagcctgggcgacagagcaagactctgccaaaaaaaaaaaaaaggagagagagagatgatgattttttatctttgaagatacgccaatgaaactggattttcccccaggtcagagaggc

SEQ ID NO: 10 - DNA, 773 bp, Homo sapiens
  misc_difference (1)..(773): /note=“rs11031755”
caaattcaatttatacaatcatctcagttaacaggccattccttttcccgatattttgagagaattcatgtaataaagggattatttatagtgccgtcgaaagagcattgacttgggaggaaaacagaactgctcttgatgtgagttggccactcaccagccttatgacctagcaaattatttcatctctctttcagtatcttccacatcactgagcaagtggatggagagaggaaagacacctggtgaatcccggtgataagactgatgattggaaataaaatttgccatgaaatgggacaagcatactccagtatgactgyatcacaattaattgcatctgcaatgaccttattcccaaataaagtcacattctgaggtgacgtgaattttggaatgacactatccagaccacacacctatattcattcatatttggaaactccatttcgggttatgaaacacgtgcctcacttgttgcatgccctttgcagacagggtgatgcaaagtgtgacacagtgtgacctgggttccagtcattgtcctgccttgtgctagatgaccctgggcaatctaattcatctttctaggcctcatccaaataatgaagtcaggagaggtggtctctaagaacccttggaatttaattattcacaagcttttgggactcaattaaacattttttttctttcttttttttttttttttttttttttttttcaggggaaatgtaggaatgagccatcatgaaacgatgccagaggctggagag

SEQ ID NO: 11 - DNA, 1448 bp, Homo sapiens
  misc_difference (1)..(1448): /note=“rs11031747”
catcagttagcatgaaccaaatacagatggggaaaataactaccctgagtctgctcactcctgcaattctaggatcagacatcccaggtgaggagtccccatgaagcggcatcgtttgtctggggtaatacctgaggttggttgcctcatgccaaggaaatcaaggacacggacacacgtggagtgaagattaagaatggaggtttaataggcgaaattaagaaaaaagagaaatagctctcccttctgcagagagagaggagatcccgagggggtcttctggtttggtggtgaaatgcacagggttttatagtctggtttgcggaggctgtgtctgacttacatagggcccaaagattggttggaccaggcatgacatttatacagcccgtgaagaagttggccaccccaccctaatcttttgttatgcaaatgagtttttttaactggccagcaccacattgtcagctccttactgtacacatggttgataaaggaaagggaagatggagcctccatgttggacatgtctagcccccaggtagccttttcctattgtcacagctgcaggcattcacctctgcaagcttccgacttgcttatctatgtttgcagctcaattttacaggctgctttttgttagaaaagaaatgatttggccgggcgcagtggctcatgcctgtaatcccagcactttgggaggccgaggcaggcggatcacctgaggtcaggagttcaaggcccygtctctctactaaaaatacaaaaattagccgggcgtgggggccggcacctgtaattccagctacttggaggctgaggcaggagaatcgcttgaatccggaaggcggaggttgcagtgagccgagatggcgccactgcacttcagtctaggcaacaagagcgaaactccgtctcaaaaaaaaaaaaaaaaaaacactggggggctgctctttatgaaaagggaaaccttaccaaggacttccttatcctcactatctgtctaaataatttcttcttaactcctgtatcacccagatatctccgtgaggagtcaggagggaaatttgggctgattgctgggtgtttttgtttttgtttgtttttggttttttttgagacggagtctcgggcagctacccaggctggagtgcagtggtgcaatctcggctcactgcaagctccgcctcctgggttcacgccattcttctgcctcagcctccggagtagcagggactacaggcatccgccaccacgcctggctaattttttgtgtttttagtagagacggggtttcaccgtgttagccaagatggtctctatctactgacctcgtgatccgcccccctcagcctcccaaagtgctggaattacaggcatgagccaccgaccccggcccaggctggtttgtttttaatgagaggctgggtggctgttagggaggggga

SEQ ID NO: 12 - DNA, 1448 bp, Homo sapiens
  misc_difference (1)..(1448): /note=“rs3858439”
tccccctccctaacagccacccagcctctcattaaaaacaaaccagcctgggccggggtcggtggctcatgcctgtaattccagcactttgggaggctgaggggggcggatcacgaggtcagtagatagagaccatcttggctaacacggtgaaaccccgtctctactaaaaacacaaaaaattagccaggcgtggtggcggatgcctgtagtccctgctactccggaggctgaggcagaagaatggcgtgaacccaggaggcggagcttgcagtgagccgagattgcaccactgcactccagcctgggtagctgcccgagactccgtctcaaaaaaaaccaaaaacaaacaaaaacaaaaacacccagcaatcagcccaaatttccctcctgactcctcacggagatatctgggtgatacaggagttaagaagaaattatttagacagatagtgaggataaggaagtccttggtaaggtttcccttttcataaagagcagccccccagtgtttttttttttttttttttgagacggagtttcgctcttgttgcctagactgaagtgcagtggcgccatctcggctcactgcaacctccgccttccggattcaagcgattctcctgcctcagcctccaagtagctggaattacaggtgccggcccccacgcccggctaatttttgtatttttagtagagagacagggccttgaactcctgacctcaggtgatccgcctgcctcggcctcccaaagtgctgggattacaggcatgagccactgcgcccggccaaatcatttcttttctaacaaaaagcagcctgtaaaattgagctgcaaacatagataagcaagtcggaagcttgcagaggtgaatgcctgcagctgtgacaataggaaaaggctacctgggggctagacatgtccaacatggaggctccatcttccctttcctttatcaaccatgtgtacagtaaggagctgacaatgtggtgctggccagttaaaaaamctcatttgcataacaaaagattagggtggggtggccaacttcttcacgggctgtataaatgtcatgcctggtccaaccaatctttgggccctatgtaagtcagacacagcctccgcaaaccagactataaaaccctgtgcatttcaccaccaaaccagaagaccccctcgggatctcctctctctctgcagaagggagagctatttctcttttttcttaatttcgcctattaaacctccattcttaatcttcactccacgtgtgtccgtgtccttgatttccttggcatgaggcaaccaacctcaggtattaccccagacaaacgatgccgcttcatggggactcctcacctgggatgtctgatcctagaattgcaggagtgagcagactcagggtagttattttccccatctgtatttggttcatgctaactgatg

SEQ ID NO: 13 - DNA, 1548 bp, Homo sapiens
  misc_difference (1)..(1548): /note=“rs3858438”
gcctctcattaaaaacaaaccagcctgggccggggtcggtggctcatgcctgtaattccagcactttgggaggctgaggggggcggatcacgaggtcagtagatagagaccatcttggctaacacggtgaaaccccgtctctactaaaaacacaaaaaattagccaggcgtggtggcggatgcctgtagtccctgctactccggaggctgaggcagaagaatggcgtgaacccaggaggcggagcttgcagtgagccgagattgcaccactgcactccagcctgggtagctgcccgagactccgtctcaaaaaaaaccaaaaacaaacaaaaacaaaaacacccagcaatcagcccaaatttccctcctgactcctcacggagatatctgggtgatacaggagttaagaagaaattatttagacagatagtgaggataaggaagtccttggtaaggtttcccttttcataaagagcagccccccagtgtttttttttttttttttttgagacggagtttcgctcttgttgcctagactgaagtgcagtggcgccatctcggctcactgcaacctccgccttccggattcaagcgattctcctgcctcagcctccaagtagctggaattacaggtgccggcccccacgcccggctaatttttgtatttttagtagagagacagggccttgaactcctgacctcaggtgatccgcctgcctcggcctcccaaagtgctgggattacaggcatgagccactgcgcccggccaaatcatttcttttctaacaaaaagcagcctgtaaaattgagctgcaaacatagataagcaagtcggaagcttgcagaggtgaatgcctgcagctgtgacaataggaaaaggctacctgggggctagacatgtccaacatggaggctccatcttccctttcctttatcaaccatgtgtacagtaaggagctgacaatgtggtgctggccagttaaaaaaactcatttgcataacaaaagattagggtggggtggccaacttcttcacgggctgtataaatgtcatgcctggtccaaccaatctttgggccctatgtaagtcagacacagcctccgcaaaccagactataaaaccctgtgcatttcaccaccaaaccagaagaccccctcgggatctcctctctctctgcagaagggagagctatttctcttttttcttaatttcgcctattaaacctccattcttaatcttcactccacgtgtgtccgtgtccttgatttccttggcatgaggcaaccaacctcaggtattaccccagacaaacgatgccgcttcatggggactcctcacctgggatgtstgatcctagaattgcaggagtgagcagactcagggtagttattttccccatctgtatttggttcatgctaactgatgcctccactttaacaggcaagccctgccccatcctttccctgttagcctgtgggactctcgtgagaacacagatacgtgtgcagctacagaaccacccttttccttgctctcttgtcatttact

SEQ ID NO: 14 - DNA, 1001 bp, Homo sapiens
  misc_difference (1)..(1001): /note=“ | rs11031746”
cttggcctcccaaagtgcggggattacaggcgtgagccactgcacccagatgagattgagacttagatttaggactgtcaggggtcggcagttggctggatgttgggtgaggattacagaaggaggagtcccaggtttctggcttgagctacatggggaagaaggccatggttcctgttttgaacatgttgtcttggtgcctaggagagatcggagtggactgcctaggaaacagctggctgcagctctgtcttggggtctggtgaggattaaacaaatgaagtatggaaagcacttagcccagtggctggcacatagtccgtacttaataaatatgaaatattattattatgtattagttttatattcaggtctgtgctgtaccttatcaaattatgccctgagagccatttggagcaaaataaaaagaaggcaagtaaatgacaagagagcaaggaaaagggtggttctgtagctgcacacgtatctgtgttctcacgmgagtcccacaggctaacagggaaaggatggggcagggcttgcctgttaaagtggaggcatcagttagcatgaaccaaatacagatggggaaaataactaccctgagtctgctcactcctgcaattctaggatcagacatcccaggtgaggagtccccatgaagcggcatcgtttgtctggggtaatacctgaggttggttgcctcatgccaaggaaatcaaggacacggacacacgtggagtgaagattaagaatggaggtttaataggcgaaattaagaaaaaagagaaatagctctcccttctgcagagagagaggagatcccgagggggtcttctggtttggtggtgaaatgcacagggttttatagtctggtttgcggaggctgtgtctgacttacatagggcccaaagattggttggaccaggcatgacatttatacagcccgtgaagaagttggccaccccaccctaatcttttgttatgcaaatgagtttttt

SEQ ID NO: 15 - DNA, 1153 bp, Homo sapiens
  misc_difference (1)..(1153): /note=“rs11031745”
atgaagtagatctttggagctgggagtcactgagcaagttgttacaaggctgcaaggtaaggaccaggcatgaaaggcctgggaagccctgggaagtttaggctttaacctaagggcaaagggtggcaaggtgagatggaaaagtcacttggctcctatgccagtccagattgaagggctggtgatggagagaatagccaggaggtatgatagtaatccagagacagatgctgggactaggagggctggggacaggtggaggcatgaggagagattgagatttatttatttgttttattattattattctttgagacacagtctccctctgtcgcccaggctggagtgcagtggcactatctcagctcactgcaacctctgcctccctggttggagcaattcttatgcctcagcctcctgagtaatcctcagctgggattacaggcgtgcgtcaccatgcccggctaatttttttgtatttttaatagagatggggtttcaccatgtttcccaggctggtctcaaactcctgggctcaagtgatccacctgccttggcctcccaaagtgcggggattacaggcgtgagccactgcacccagatgagattgagacttagatttaggactgtcaggggtcggcagttggctggatgttgggtgaggattacagaaggaggagtcccaggtttctggcttgagctacatggggaagaaggccatggttcctgttttgaacatgttgtcttggtgcctaggagagatcggagtggactgcctaggaaacagctggctgcagctctgtcttggggtctggtgaggattaaacaaatgaagtatggaaagcacttagcccagtggctggcacatagtccgtacttaataaatatgaaatattattattatgtattagttttatattcaggtctgtgctgtaccttatcaaattatgccytgagagccatttggagcaaaataaaaagaaggcaagtaaatgacaagagagcaaggaaaagggtggttctgtagctgcacacgtatctgtgttctcacgagagtcccacaggctaacagggaaaggatggggcagggcttgcctgttaaagtggaggcatcagttagcatgaaccaaatacagatggggaaaataactac

SEQ ID NO: 16 - DNA, 1105 bp, Homo sapiens
  misc_difference (1)..(1105): /note=“rs11031744”
agtttagaacaaaggcttaaaagtcacagagaatgaagtagatctttggagctgggagtcactgagcaagttgttacaaggctgcaaggtaaggaccaggcatgaaaggcctgggaagccctgggaagtttaggctttaacctaagggcaaagggtggcaaggtgagatggaaaagtcacttggctcctatgccagtccagattgaagggctggtgatggagagaatagccaggaggtatgatagtaatccagagacagatgctgggactaggagggctggggacaggtggaggcatgaggagagattgagatttatttatttgttttattattattattctttgagacacagtctccctctgtcgcccaggctggagtgcagtggcactatctcagctcactgcaacctctgcctccctggttggagcaattcttatgcctcagcctcctgagtaatcctcagctgggattacaggcgtgcgtcaccatgcccggctaatttttttgtatttttaatagagatggggtttcaccatgtttcccaggctggtctcaaactcctgggctcaagtgatccacctgccttggcctcccaaagtgcggggattacaggcgtgagccactgcacccagatgagattgagacttagatttaggactgtcaggggtcggcagttggctggatgttgggtgaggattacagaaggaggagtcccaggtttctggcttgagctacatggggaagaaggccatggttcctgttttgaacatgttgtcttggtgcctaggagagatcggagtggactgcctaggaaacagctggctgcagctctgtcttggggtctggtgaggattaaacaaatgaagtatggaaagcacttagcccagtggctggcacatagtcygtacttaataaatatgaaatattattattatgtattagttttatattcaggtctgtgctgtaccttatcaaattatgccctgagagccatttggagcaaaataaaaagaaggcaagtaaatgacaagagagcaaggaaaagggtggttctgtagctgcacacgtatctgtgttctcacgagagtcccacaggctaacagg

SEQ ID NO: 17 - DNA, 1053 bp, Homo sapiens
  misc_difference (1)..(1053): /note=“rs3858436”
tgctctcttgtcatttacttgccttctttttattttgctccaaatggctctcagggcataatttgataaggtacagcacagacctgaatataaaactaatacataataataatatttcatatttattaagtacggactatgtgccagccactgggctaagtgctttccatacttcatttgtttaatcctcaccagaccccaagacagagctgcagccagctgtttcctaggcagtccactccgatctctcctaggcaccaagacaacatgttcaaaacaggaaccatggccttcttccccatgtagctcaagccagaaacctgggactcctccttctgtaatcctcacccaacatccagccaactgccgacccctgacagtcctaaatctaagtctcaatctcatctgggtgcagtggctcacgcctgtaatccccgcactttgggaggccaaggcaggtggatcacttgagcccaggagtttgagaccagcctgggaaacatggtgaaaccccatctctattaaaaatacaaaaaaattagccgggcatggtgacgcacgcctgtaatcccagctgaggattactcaggaggctgaggcataagaattgctccaaccagggaggcagaggttgcagtgagctgagatagtgccactgcactccagcctgggcgacagagggagactgtgtctcaaagaataataataataaaacaaataaataaatctcaatctctcctcatgcctccacctgtccccagccctcctagtcccagcatctgtctctggattactrtcatacctcctggctattctctccatcaccagcccttcaatctggactggcataggagccaagtgacttttccatctcaccttgccaccctttgcccttaggttaaagcctaaacttcccagggcttcccaggcctttcatgcctggtccttaccttgcagccttgtaacaacttgctcagtgactcccagctccaaagatctacttcattctctgtgacttttaagcctttgttctaaactatctgaattctttca

SEQ ID NO: 18 - DNA, 1410 bp, Homo sapiens
  misc_difference (1)..(1410): /note=“rs2418901”
ataataactcaaagagagggccgggcacggtggctcacacctgtaatcccagtactttgggaggccaaggcaggtggatcacaaggtcaagagatcaataccgtcctggccaacatgcttaaaccccatccctactaaaaatacaaaaattagccaggtgtggtggcatgcacctgtaatcccaactactcaggaggctgaggcaggagaatcgcttgaacctgggaggtggagattgtagtgagcccagttcgcaccactgcactccaacctggcaacagagcgagactccatcaaaacaacaacaacaacaacaaaacaaagcaaaaaagcaaaaacaaaacaaacaaaaaagaaacttcaaagagagaccagctggccatttcttgtaatcacaccactccactgaggaagagaaccccaggactcactgctagagtagggaggtggggagcaaaatgctaggagaaaaagagaggttaatatttcaaagatggtaccctatcgcatattattgagcacatgctatggagcagtcactgttctggatgctaaggatatagagatggatgcaatctacacaggggatgaatttgtttccaattgttgctataaaaaattaccttgtggattaaaacaacacaaatttcttatagttttggagatcagcagtccaaaatggttctcactgggctaaaatcgaggtatcaacagggccactttcctttcggaagatctaggggatgatgcattttcacaccttctccagctgcccacatttcttggctcgtggcctgtatcactccgagctttgcttcctccatcacatctccttctctgatcctcatgctcctgctccccactctctgctttataaggacccttrttacatttagagtccacctccagaatccaggaaaatctcccaatcttaaaatccttagcttaatcacatcagcaaagtcccctttgccttgtatcctgggttctagggattaggacatggacatctgtgggaagacattaatctgcctactacaggggatttgtctagaggtgctgtggtaagccctaccacaggagaaatgtagggtgtatgagagtttgcaggaatgccagatttggcgatcaggaaaagctcctctgtttagtctgagacctggaggatgaataagaaggatttgggaggccgggtgtggtggctcatgcttgtaatcccagctacttgggaggctgaggcaggagaatcacttgaacccaagaggtgaggttgcagtgagctgagatcgagccactgcattccagcctgggcgacagagcaagcctctgcctcaaaaaaaaaaaaaaaaaaaaaagaaggatttgggagacatgaggaagaggaagttatactgaaagaattcagatagt